(19) United States
(12) Patent Application Publication
(10) Pub. No.: US 2015/0289967 A1
(43) Pub. Date: Oct. 15, 2015

(54) NANOCOMPOSITES FOR SOFT TISSUE REPAIR AND REPLACEMENT

(71) Applicant: The Curators of the University of Missouri, Columbia, MO (US)

(72) Inventors: Sheila Ann Grant, Columbia, MO (US); David Grant, Columbia, MO (US); Richard A. White, Columbia, MO (US); Sharon Bachman, Columbia, MO (US); Daniel Grant, Columbia, MO (US); Matthew Cozad, Minneapolis, MN (US)

(21) Appl. No.: 14/387,092

(22) PCT Filed: Mar. 15, 2013
(86) PCT No.: PCT/US2013/031908

§ 371 Date: Sep. 22, 2014

Related U.S. Application Data
(60) Provisional application No. 61/685,685, filed on Mar. 22, 2012.

(51) Int. Cl. A61F 2/08; A61F 2/30

(52) U.S. Cl. CPC A61F 2/08; A61F 2/30756; A61F 2230/0069; A61F 2240/001

(57) ABSTRACT

The present invention relates to nanocomposites, methods of fabrication, and applications thereof. More specifically, the present invention relates to a series of biocompatible materials that can be used in soft tissue repair and replacement, particularly, for ligament, tendon, and cartilage repair and replacement in a living body.

### FIELD OF THE INVENTION

The present invention relates to nanocomposites, methods of fabrication, and applications thereof More specifically, the present invention relates to a series of biocompatible materials that can be used in soft tissue repair and replacement, particularly, for ligament, tendon, and cartilage repair and replacement in a living body.

### BACKGROUND OF THE INVENTION

There is a clinical need in the orthopedic field driven by the failure rate of soft tissue replacements for tendons, ligaments, and cartilage repair and replacement. A problem currently experienced by many allografts is the lack of cellular integration and remodeling, leading to graft deterioration. Thus, there is a need for new graft materials that promote cellularity, integration of the graft with the surrounding tissue, and recapture of the natural joint function.

The soft tissue market is composed of three main areas: hernia, women's health, and orthopedic. All three of these areas utilize soft tissue implants (autografts, allografts, and xenografts) that are designed to fix a tissue defect and promote new tissue growth as the implant degrades into the body; however, these graft materials have a high failure rate.

Thus, a need exists for new and improved soft tissue implant materials that combats the problems of infection and inflammation, while promoting tissue integration and improving the overall biocompatibility with the surrounding tissue and bone when used in soft tissue repair.

### SUMMARY OF THE INVENTION

An aspect of the invention is an oblong or elongate nanocomposite comprising: an oblong or elongate decellularized tissue substrate, a first nanomaterial crosslinked with the decellularized tissue substrate and adapted to promote tissue ingrowth, and a second nanomaterial crosslinked with the decellularized tissue substrate and adapted to promote osseointegration. The decellularized tissue substrate having a length, first and second longitudinal end margins, and a central longitudinal portion intermediate the first and second longitudinal end margins. The decellularized tissue substrate can include extracellular matrix components and be substantially free from cells and cellular remnants. The first nanomaterial has a substantially uniform distribution of mass concentration along the length of the decellularized tissue substrate. The second nanomaterial has a non-uniform distribution of mass concentration along the length of the decellularized tissue substrate that is non-uniform, wherein the mass concentration of the second nanomaterial is greater adjacent at least one of the first and second longitudinal end margins of the decellularized tissue substrate than at the central longitudinal portion of the decellularized tissue substrate.

Another aspect is an oblong or elongate nanocomposite comprising: an oblong or elongate tissue substrate, a first nanomaterial crosslinked with the tissue substrate and adapted to promote tissue ingrowth, and a second nanomaterial crosslinked with the tissue substrate and adapted to promote osseointegration. The tissue substrate having a length, first and second longitudinal end margins, and a central longitudinal portion intermediate the first and second longitudinal end margins. The first nanomaterial has a substantially uniform distribution of mass concentration along the length of the tissue substrate. The second nanomaterial has a non-uniform distribution of mass concentration along the length of the tissue substrate that is non-uniform, wherein the mass concentration of the second nanomaterial is greater adjacent at least one of the first and second longitudinal end margins of the tissue substrate than at the central longitudinal portion of the tissue substrate. The tissue substrate can be an autograft and is harvested from a subject that will have the nanocomposite implanted in their body.

Another aspect of the invention is the use of the oblong or elongate nanocomposite as described herein for soft tissue repair or replacement. The soft tissue repair or replacement can be a ligament, tendon, or cartilage repair or replacement.

The nanocomposites described herein can also be used for ligament or tendon repair or replacement.

Yet another aspect is a method for treating a soft tissue injury comprising implanting an oblong or elongate nanocomposite as described herein at the site of the injury in a subject.

A further aspect is a method for producing an oblong or elongate nanocomposite, comprising decellularizing a selected biological tissue substrate to produce an oblong or elongate decellularized tissue substrate with cells and cellular remnants removed but extracellular matrix components intact; functionalizing a selected first nanomaterial adapted to promote tissue ingrowth to produce a functionalized first nanomaterial with surface functional groups capable of bonding with the decellularized tissue substrate; selecting a second nanomaterial adapted to promote osseointegration; and crosslinking the decellularized tissue substrate with the functionalized first nanomaterial and the second nanomaterial by contacting the whole oblong or elongate decellularized tissue substrate with the functionalized first nanomaterial and contacting at least one longitudinal end margin of the oblong or elongate decellularized tissue substrate with the second nanomaterial to form the oblong or elongate nanocomposite.

Another aspect is a method for producing an oblong or elongate nanocomposite, comprising selecting a tissue substrate from a subject's tissue to produce an oblong or elongate tissue substrate; functionalizing a selected first nanomaterial adapted to promote tissue ingrowth to produce a functionalized first nanomaterial with surface functional groups capable of bonding with the tissue substrate; selecting a second nanomaterial adapted to promote osseointegration; and crosslinking the tissue substrate with the functionalized first nanomaterial and the second nanomaterial by contacting the whole oblong or elongate tissue substrate with the functionalized first nanomaterial and contacting at least one longitudinal end margin of the oblong or elongate tissue substrate with the second nanomaterial to form the oblong or elongate nanocomposite.

Other objects and features will be in part apparent and in part pointed out hereinafter.

### BRIEF DESCRIPTION OF THE DRAWINGS

FIGS. 1A and 1B are schematic diagrams of the nanocomposites wherein the tissue substrate is represented as a cylinder, but could be the desired shape of the tissue substrate and the smaller spheres represent the first nanomaterial and the larger spheres represent the second nanomaterial.

FIG. 2 is the Scanning Electron Micrograph (“SEM”) of decellularized tissue with gold nanoparticles (AuNP) conjugated throughout the graft and hydroxyapatite nanoparticles (HaNP) conjugated primarily at the ends of the graft.

FIG. 3 is the SEM of silver nanoparticles (AgNP) conjugated to decellularized tissue.

FIG. 4 is the SEM of silver nanowires (AgNW) conjugated to decellularized tissue.

FIG. 5 is the SEM of calcium oxide (“CaO”) nanoparticles incubated with L929 murine fibroblast cells.

FIG. 6 is a SEM displaying HaNP and AuNP conjugated to an acellular graft.

FIG. 7 is a graph of DNA quantification assays used to determine cellular proliferation.

FIG. 8A is a confocal microscopy image of a crosslinked allograft showing a very slight amount of fluorescence.

FIG. 8B is a confocal microscopy image of a crosslinked xenograft showing a very slight amount of fluorescence.

FIG. 8C is a confocal microscopy image of a HaNP/AuNP-allograft showing some green fluorescence protein (GFP) infiltration.

FIG. 8D is a confocal microscopy image of another HaNP/AuNP-allograft showing some GFP infiltration.

FIG. 8E is a confocal microscopy image of an AuNP-allograft showing GFP cellular highways into the graft.

Corresponding reference characters indicate corresponding parts throughout the drawings.

### DESCRIPTION OF THE PREFERRED EMBODIMENTS

The inventive nanocomposite involves crosslinking nanomaterials to decellularized tissues, which improves the overall strength of the material while promoting tissue in-growth when utilized for soft tissue repair. The first nanomaterial crosslinked to the decellularized tissue substrate promotes tissue ingrowth to remodel the tissue around the nanocomposite and has a substantially uniform distribution of mass concentration along the length of the decellularized tissue substrate. The second nanomaterial crosslinked to the decellularized tissue substrate promotes osseointegration to grow bone cells into the nanocomposite and has a non-uniform distribution of mass concentration along the length of the decellularized tissue substrate, wherein the mass concentration of the second nanomaterial is greater adjacent at least one of the first and second longitudinal end margins of the decellularized tissue substrate than at the central longitudinal portion of the decellularized tissue substrate. This arrangement of nanomaterials on the decellularized tissue substrate provides an oblong or elongate nanocomposite that can promote tissue growth along its full length while inducing bone growth into the nanocomposite on one or more longitudinal ends. This arrangement makes the oblong or elongate nanocomposites particularly suited for ligament, tendon, and cartilage repair or replacement applications.

The invention uses decellularized tissues as the biologic substrates upon which nanomaterials are crosslinked for ligament, tendon, and cartilage repair and replacement. These nanocomposites are advantageous in comparison to the cadaver tissue currently used since the nanomaterials improve the performance of the nanocomposites by increasing tissue ingrowth and neovascularization of the implant by the surrounding tissue. Also, the decellularized tissue includes a mixture of collagen, elastin, and other structural and functional proteins that constitute the extracellular matrix. The extracellular matrix (“ECM”) is an ideal substrate material because it naturally possesses the bioactive components and structure necessary to support cell adhesion and tissue ingrowth, initiate angiogenesis, and promote constructive tissue regeneration. As ECM scaffolds degrade, growth factors and peptides are released. These elements possess antimicrobial properties that ward off potential pathogens, and they also influence angiogenesis and tissue remodeling through the recruitment of endothelial and bone marrow-derived cells.

### Nanocomposites

The nanocomposites of the invention are oblong or elongate structures, typically cylinders, rectangles, bands, ovals, elipses, or the shape of the desired ligament, tendon, or cartilage to be repaired or replaced that can have a range of dimensions depending on the desired use. For example, the decellularized tissue substrate can be cut to fit the particular site either before or after crosslinking to the nanomaterials. Thus, the nanocomposite can be a range of dimensions.

Generally, the nanocomposite can be dimensioned to be compatible with various ligaments and tendons.

These ligaments and tendons are typically involved with various joints. These joints include the knee, elbow, hip, ankle, wrist, shoulder, and the like.

When the decellularized tissue is an anterior tibialis tendon (used for anterior cruciate ligament (ACL) reconstruction) or a gracilis tendon, the tendon is greater than 20 cm in length by greater than 2 cm in width. When using a posterior tibialis tendon or a peroneus longus tendon, the length of the tendon is greater than 22 cm and the width is about 2 cm. When using a semitendinosus tendon, the length can be greater than or equal to 26 cm or less than 26 cm and be about 2 cm in width.

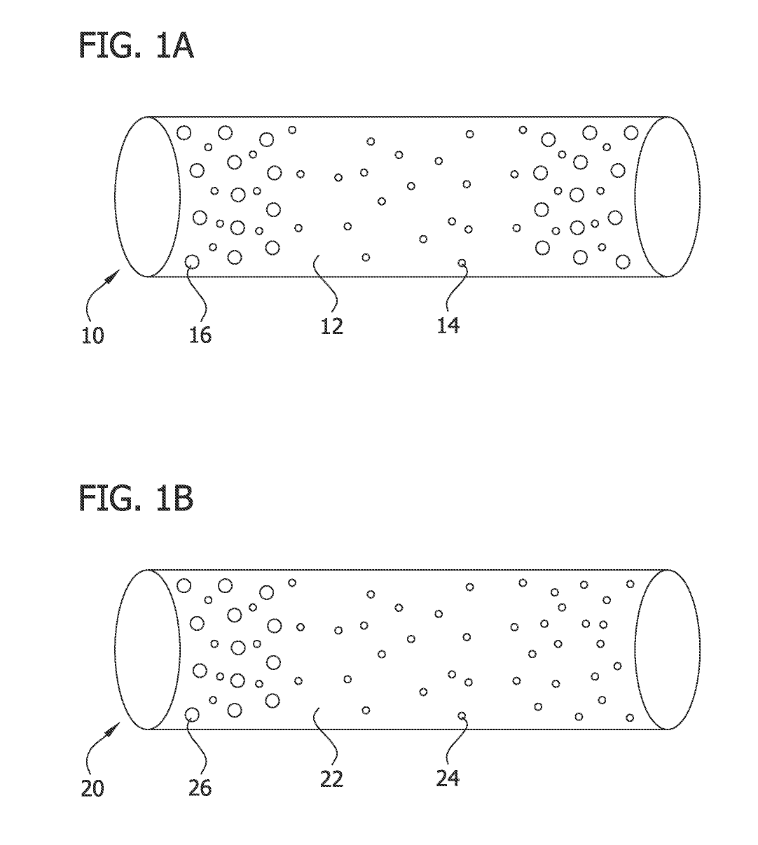

FIG. 1A is directed to a nanocomposite 10 comprising a tissue substrate or a decellularized tissue substrate 12, a first nanoparticle adapted to promote tissue ingrowth 14 deposited uniformly on the tissue substrate or decellularized tissue substrate, and a second nanoparticle adapted to promote osseointegration 16 deposited in a manner that a mass concentration of the second nanomaterial is greater adjacent both of the first and second longitudinal end margins of the tissue substrate or decellularized tissue substrate than at the central longitudinal portion of the tissue substrate or decellularized tissue substrate. The tissue substrate or decellularized tissue substrate is shown as a cylinder, but a person of ordinary skill would understand that the tissue substrate or decellularized tissue substrate could have the desired shape and dimensions for the particular use of the nanocomposite.

FIG. 1B is directed to a nanocomposite 20 comprising a tissue substrate or decellularized tissue substrate 22, a first nanoparticle adapted to promote tissue ingrowth 24 deposited uniformly on the tissue substrate or decellularized tissue substrate, and a second nanoparticle adapted to promote osseointegration 26 deposited in a manner that a mass concentration of the second nanomaterial is greater adjacent the first longitudinal end margin of the tissue substrate or decellularized tissue substrate than at the central longitudinal portion of the tissue substrate or decellularized tissue substrate. Again, a person of ordinary skill would understand that the tissue substrate or decellularized tissue substrate could have the desired shape and dimensions for the particular use of the nanocomposite.

Further, the nanoparticles, nanowires, nanofibers, or nanorods distributed on the surface and/or within the decellularized tissue substrate either uniformly or nonuniformly depending on their function. The first nanomaterial crosslinked to the decellularized tissue substrate promotes tissue ingrowth and has a substantially uniform distribution of mass concentration along the length of the decellularized tissue substrate. The second nanomaterial crosslinked to the decellularized tissue substrate promotes osseointegration and has a non-uniform distribution of mass concentration along the length of the decellularized tissue substrate; this mass concentration is greater adjacent at least one of the first and second longitudinal end margins of the decellularized tissue substrate than at the central longitudinal portion of the decellularized tissue substrate.

The oblong or elongate nanocomposite of the invention can have a concentration of the second nanomaterial that is higher toward the first and second longitudinal end margins of the decellularized tissue substrate.

Additionally, the oblong or elongate nanocomposite can have a configuration where the second nanomaterial is substantially absent from the central longitudinal portion of the decellularized tissue substrate.

Further, the oblong or elongate nanocomposite can have a first longitudinal end margin that is at least 30% of the total length of the decellularized tissue substrate measured from the first longitudinal end of the decellularized tissue substrate. The first longitudinal end margin can also be at least 25% of the total length of the decellularized tissue substrate measured from the first longitudinal end of the decellularized tissue substrate.

The oblong or elongate nanocomposite can also have the central longitudinal portion comprise at least 40% of the total length of the decellularized tissue substrate and be symmetrical about a longitudinal center of the decellularized tissue substrate. The central longitudinal portion can also comprise at least 50% of the total length of the decellularized tissue substrate and the central longitudinal portion and be symmetrical about a longitudinal center of the decellularized tissue substrate.

The mass concentration of the first and second nanomaterials on the surface of the decellularized surface and/or within the decellularized tissue substrate can be optimized to provide the appropriate surface area for cell growth, infiltration, and vascularization. Nanoparticles having a mean diameter of from about 15 nm to about 200 nm, from about 20 nm to about 200 nm, or from about 20 nm to about 150 nm can be used to provide a surface for cell growth.

The mechanical and chemical properties of the nanocomposites desirably do not change significantly once implanted in an animal. For example, the viscoeslasticity of the nanocomposite does not change significantly as cells from the surrounding tissue infiltrate the nanocomposite and it degrades. In order to have a composite that has a desired viscoelasticity, the material should have an appropriate degradation rate. Further, the viscoelasticity can be measured by the Young's modulus wherein a higher value means the material is stiffer and a lower value means the material is less stiff. Preferably, the viscoelasticity of the nanocomposite is from about 1.2 GPa to about 1.8 GPa.

Depending on the chemical identity of the nanoparticles that are crosslinked to the decellularized tissue substrate, the nanocomposite can scavenge free radicals. For example, gold nanoparticles, gold nanorods, and gold nanofibers have the ability to scavenge free radicals. Without being bound by theory, it is believed that the free radical scavenging ability of the gold nanoparticles is able to ameliorate and/or reduce inflammation at the nanocomposite implant site. The free radical scavenging capability of the gold nanoparticle nanocomposite can be measured using the technique of Hsu et al., J. Biomedical Materials Research Part A 2006, 759. The capacity of the sample to scavenge can be measured by placing the sample (7.5 mm diameter, 1 mm thick) in 3 mL of 32 μM 2,2-diphenyl-1-picrylhydrazyl (DPPH), vortexed, and left to stand at room temperature for 90 minutes. The absorbance of the reaction mixture can be measured at 515 nm using a UV/VIS spectrophotometer and the following equation:

Scavenging ratio (%)=[1−Absorbance of test sample/Absorbance of control]×100%.

Additionally, silver nanoparticles are free radical scavengers. Hsu et al., Biomaterials 2010, 31, 6796.

Thus, the free radical scavenging ratio of the gold nanoparticle nanocomposite or of the silver nanoparticle nanocomposite is expected to be higher than the scavenging ratio of the decellularized tissue substrate without gold nanoparticles.

When the nanocomposite is implanted at a desired site in an animal, typically there is a layer of muscle or bone next to the nanocomposite implant next to another layer of tissue. Thus, immediately after the placement of the implant until the time that the implant has been completely absorbed by the body, these three layers will be present. Over time, the overlying tissue will migrate and infiltrate the implant and the border between the implant and the tissue will be compromised.

The biodegradability of the implant is usually determined by removing the implant and surrounding tissue from the animal and performing a visual inspection of the margins between the adjacent muscle and the implant as well as the adjacent tissue and the implant. At a certain time after placement, the margin between the tissue (muscle or other tissue) and the implant will not be visible. At this point the implant in considered completely biodegraded. Preferably, the time for complete degradation of the implant is substantially the same as the healing time for the tissue. For example, the time for degradation ranges from about 1 month to about 12 months; from about 1 month to about 9 months; from about 1 month to about 6 months; from about 2 months to about 6 months; or from about 3 months to about 6 months.

The biocompatibility, mechanical properties, and in vivo stability of the nanocomposite render it suitable for use in ligament, tendon, and cartilage repair and replacement. The composite has a supple, flexible membranous structure substantially similar to the intact biologic material from which it is produced. It is resilient so that it can be rolled, stretched or otherwise deformed in use, e.g., in the course of surgical implantation and revert to its original configuration when external forces holding the composite in the deformed configuration are removed.

Especially important to the function of the nanocomposite is its stability in vivo. It retains its suppleness and flexibility during healing of the surgical site where implanted and indefinitely thereafter until it has been integrated with surrounding tissue, or infiltrated and effectively displaced by endogenous tissue. The implanted nanocomposite is resistant to oxidation, and resistant to shrinkage and/or hardening.

The Young's modulus and flexural modulus of the nanocomposite each remain between 50% and 200%, more typically between 75% and 150%, most typically between 90% and 125% of their values prior to implantation after passage of 30, 60 and 90 days. After 3 months, 6 months, 9 months or one year after implantation or until the nanocomposite is effectively displaced by endogenous tissue, the Young's modulus and flexural modulus each remain between 50% and 250%, more typically between 75% and 200%, most typically between 90% and 150%, of their values prior to implantation.

Further, the nanocomposites can have a cellular integration score of 2 or 3 on a four point grading scale for histological analysis at 1 month after implant. The oblong or elongate nanocomposite can further have a cellular integration score of 2 or 3 on a four point grading scale for histological analysis at 3 month after implant.

Additionally, the oblong or elongate nanocomposite can have a neovascularization score of 2 or 3 on a four point grading scale for histological analysis at 1 month after implant. The neovascularization score of 2 or 3 on a four point grading scale for histological analysis at 3 month after implant.

Histological scoring information is disclosed in more detail in Example 6.

When the substrate used for the oblong or elongate nanocomposite is an autograft and harvested from the patient's body, the nanocomposite can be prepared by crosslinking the desired nanomaterial to the autograft without the need for decellularizing the harvested substrate. Thus, the same nanomaterials and methods of synthesis described herein would be used with the autograft tissue substrate instead of the decellularized tissue substrate.

### Decellularized Tissue Substrate

The decellularized tissue substrate may be obtained from treatment of biological tissue, which may be harvested from either allograft or xenograft. The tissue is decellularized in that cells and cellular remnants are removed while the extracellular matrix components remains intact. A variety of biological tissue donor sources may be employed, such as human (ligament, tendon, cartilage, skin, small intestine submucosa, pericardium, or bladder), porcine (diaphragm, skin, small intestine submucosa, pericardium, or bladder), bovine (diaphragm, skin, small intestine submucosa, pericardium, or bladder), and equine (diaphragm, skin, small intestine submucosa, pericardium, or bladder). Many of these materials provide desirable degradation characteristics and when implanted either alone or once crosslinked to nanoparticles, can release growth factors and peptides that possess antimicrobial properties, enhance angiogenesis, and aid tissue remodeling by attracting endothelial and bone marrow-derived cells to the implant site.

In many instances, the tissue may be selected according to its handling properties for surgical manipulation and mechanical properties (strength, elasticity, size, etc.) required for the targeted soft tissue repair application.

Generally, the nanocomposite can be dimensioned to be compatible with various ligaments and tendons.

These ligaments and tendons are typically involved with various joints. These joints include the knee, elbow, hip, ankle, wrist, shoulder, and the like.

When the decellularized tissue is an anterior tibialis tendon (used for anterior cruciate ligament (ACL) reconstruction) or a gracilis tendon, the tendon is greater than 20 cm in length by greater than 2 cm in width. When using a posterior tibialis tendon or a peroneus longus tendon, the length of the tendon is greater than 22 cm and the width is about 2 cm. When using a semitendinosus tendon, the length can be greater than or equal to 26 cm or less than 26 cm and be about 2 cm in width.

Further, diaphragm, depending upon the species harvested from can be greater than 10 cm in diameter and used in ligament, tendon, or cartilage repair or replacement. Small intestine submucosa and skin can be available in various sizes and can be utilized in various ligament, tendon, or cartilage repair or replacement applications.

Also, the tensile strength of the decellularized tissue substrate measured at yield ranges from about 50 MPa to about 150 MPa, from about 60 MPa to about 140 MPa; from about 70 MPa to about 130 MPa; from about 80 MPa to about 120 MPa; or from about 90 MPa to about 110 MPa. For commercialization purposes, a user may also consider whether large quantities of the tissue can be easily obtained and processed.

In addition to these considerations, the degradation rate of the tissue can also influence the selection of a particular tissue. When utilized for soft tissue repair and reconstruction, it is important that the selected natural tissue is degraded by the body at a rate that matches the healing rate of the defective area so that it can serve as an effective repair material without inciting a chronic inflammatory response.

The selected biological tissues, if allografts or xenografts, need to be processed to remove native cells, i.e. “decellularized” in order to prevent an immune response when it is utilized as a soft tissue repair material. (Gilbert et al. Decellularization of tissues and organs. Biomaterials 2006; 27:3675-3683) The decellularization process may be optimized for each species and type of tissue. Successful decellularization is characterized by the removal of cellular nuclei and remnants with the retention of natural extracellular matrix components (collagen, elastin, growth factors, etc.) and overall tissue structure (collagen architecture). (Gilbert et al.) For example, from about 80% to 100%, from about 85% to about 100%, from about 90% to about 100%, or from about 95% to about 100% of the cellular nuclei and remnants are removed from the tissue. Further, the decellularized material can contain from about 0.1% to about 20%; from about 0.1% to about 15%; from about 0.1% to about 10%; from about 0.1% to about 5% of the original cellular material after decellularization. The collagen structure is ideal for cell attachment and infiltration. Thus, maintaining the collagen structure is desirable during the decellularization process. For example, the collagen structure has pore size from about 1 nm to about 100 nm. Further, the collagen structure has a porosity of from about 10% to about 90%; from about 20% to 90%; from about 30% to about 90%; from about 30% to about 80%; or from about 40% to about 80%.

The decellularized tissue substrate alone or in the nanocomposite retains its proteins, growth factors, and other peptides. For example, the decellularized tissue substrate retains growth factors such as vascular endothelial growth factor (VEGF), transforming growth factor (TGF-B1), proteins such as collagen, elastic, fibronectin, and laminin, and other compounds such a glycosaminoglycans. Because the decellularization process does not remove these proteins, growth factors, and other peptides, the tissue or nanocomposite comprising the decellularized tissue substrate can release these factors during its remodeling and resorption by the body. This release is advantageous to cell growth and cell infiltration into the affected tissue. Therefore, retention of these compounds is advantageous for the implant material.

The decellularizing process can take the form of physical (sonication, freezing, agitation, etc.), chemical (acids, ionic, non-ionic, and zwitterionic detergents, organic solvents, etc.), and enzymatic (protease, nuclease, etc.) treatments or a combination thereof and may employ any procedure commonly practiced in the field. (Gilbert et al.) Physical methods for decellularization include freezing, direct pressure, sonication, and agitation; these methods need to be modified depending on the particular tissue. Chemical methods include treatment with an acid, a base, a non-ionic detergent, an ionic detergent, a zwitterionic detergent, an organic solvent, a hypotonic solution, a hypertonic solution, a chelating agent, or a combination thereof.

The acid or base solubilizes cytoplasmic components of cells and disrupts nucleic acids. Exemplary acids and bases are acetic acid, peracetic acid, hydrochloric acid, sulfuric acid, ammonium hydroxide, or a combination thereof.

Treatment with non-ionic detergents disrupts lipid-lipid and lipid-protein interactions, while leaving protein-protein interactions intact. An exemplary non-ionic detergent is Triton X-100.

An ionic detergent solubilizes cytoplasmic and nuclear cellular membranes and tends to denature proteins. Exemplary ionic detergents are sodium dodecyl sulfate, sodium deoxycholate, Triton X-200, or a combination thereof.

A zwitterionic detergent treatment exhibits properties of on-ionic and ionic detergents. Exemplary zwitterionic detergents are 3-[3-cholamidopropyl)dimethylammonio]-1-propanesulfonate (CHAPS), sulfobetaine-10 (SB-10), sulfobetaine-16 (SB-16), or a combination thereof.

Tri(n-butyl)phosphate is an organic solvent that disrupts protein-protein interactions.

Chelating agents bind divalent metallic ions that disrupt cell adhesion to the extracellular matrix. Exemplary chelating agents are ethylenediamine tetraacetic acid (EDTA), ethylene glycol tetraacetic acid (EGTA), or a combination thereof.

The decellularization can also be carried out using enzymatic methods. Exemplary enzymes are trypsin, endonucleases, exonucleases, or a combination thereof. Trypsin cleaves peptide bonds on the C-side of arginine and lysine. Endonucleases catalyze the hydrolysis of the interior bonds of ribonucelotide and deoxyribonucleotide chains. Exonucleases catalyze the hydrolysis of the terminal bonds of ribonucleotide and deoxyribonucleotide chains. The decellularization can be performed by treatment with acetic acid, peracetic acid, hydrochloric acid, sulfuric acid, ammonium hydroxide, Triton X-100, sodium dodecyl sulfate, sodium deoxycholate, Triton X-200, 3-[3-cholamidopropyl)dimethylammonio]-1-propanesulfonate (CHAPS), sulfobetaine-10 (SB-10), sulfobetaine-16 (SB-16), tri(n-butyl)phosphate, EDTA, EGTA, or a combination thereof.

Generally, the decellularization process includes immersion of the desired tissue in an agent that can make the tissue acellular (i.e., the tissue contains no cells). The agent that makes the tissue acellular can be an acid, a solvent, a surface active agent, and the like. The concentration of the agent is from about 0.5% (v/v) to about 5% (v/v). In various preferred processes, the concentration of the agent is from about 1% (v/v) to about 2% (v/v). The tissue can be immersed in the agent for about 6 hours to about 36 hours; from about 12 hours to about 30 hours; from about 18 hours to about 30 hours; or from about 20 hours to 28 hours. The decellularization process was performed at room temperature.

The decellularization process can include immersion for 24 hours with agitation in the following solutions: (1) 0.1% (v/v) peracetic acid with 4% ethanol, (2) 1% (v/v) TritonX-100, (3) 1% (v/v) Triton X-100 with 1% (v/v) tributyl phosphate (TnBP), (4) 2% (v/v) TnBP, (5) 1% (v/v) TnBP, (6) 1% (w/v) sodium dodecyl sulfate (SDS), (7) 0.5% (w/v) SDS.

A combination of both physical and chemical treatments can be employed. This combination includes two substeps, decellularization and subsequent rinses. In the decellularization step, the selected biological tissue is submersed in a buffered solution containing an organic solvent, tri(n-butyl)phosphate (TnBP), with agitation, such as in an orbital shaker, for about 24 hours. The resulting tissue is then rinsed to remove residual solvent and cellular remnants. The rinsing solvents may be deionized water and about 70% ethanol consecutively for a period of time, such as about 24 hours each. The tissue:solution volume ratio is from about 1:500 to about 5:100; from about 1:200 to about 2:100; or about 1:100 throughout the decellularization and subsequent rinses.

Several tests may be employed to verify the effectiveness of the decellularization process, i.e., removal of all cells and cellular remnants such as DNA while leaving extracellular matrix (‘ECM’) components (such as collagen, elastin, fibronectin, laminin, and glycosaminoglycans) intact. For example, a standard histological staining with hematoxylin and eosin (H&E) may be performed to identify any cell nuclei remaining in the resulting tissue. For example, the decellularized material desirably will be substantially free of cell nuclei and cellular remnants. Preferably, when a representative section of the decellularized material (1 cm×1 cm) is stained with H&E, and/or with diamidino-2-phenylindole (DAPI) nuclear counterstaining, the decellularized material will have less than about 20 cell nuclei remaining and be substantially free of cellular remnants wherein substantially free of cell nuclei and cellular remnants means less than 15; less than 12; less than 10; less than 8; or less than 5 nuclei or cell remnants in the field of view of the decellularized tissue substrate.

Further, the collagen structure of the decellularized material is substantially the same as the structure of the tissue before decellularization. Finally, the decellularized tissue substrate is biocompatible. The biocompatibility of the tissue can be measured using flow cytometry wherein cells incubated with the decellularized tissue substrate did not show a significantly higher cell death rate as compared to the same cells under the same conditions but without contacting a tissue. A significantly higher cell death rate occurs when statistical significance (p<0.05) is measured. Microscopic analyses may be performed to verify that all fibroblasts and endothelial cells are successfully removed from the resulting tissue. Methyl green pyronin stain, which stains for DNA and RNA, may also be utilized to verify that remnants of DNA and RNA are effectively removed from the tissue during the extensive rinse sequence. Further histological analyses, such as Masson's Trichrome, Verhoeff-van Gieson, and Alcian Blue staining, may also be performed to verify that ECM components remain within the decellularized tissue substrate.

### Nanomaterials

Nanomaterials are incorporated to form the nanocomposite materials and improve the strength of the decellularized tissue substrate and its resistance to degradation by the body, as well as to influence cellular behavior and biocompatibility. Prior studies have demonstrated that nanomaterials are more hydrophilic and possess an increased number of atoms and crystal grains at their surface compared to conventional materials. The large number of grains at the surface leads to increased surface roughness, surface area, and surface energy which are thought to contribute to an increase in protein adsorption and unfolding. For example, nanoscale ceramics, metals, and polymers have all been shown to improve cellular function compared to conventional materials. Webster T J et al. J Biomed Mater Res 2000; 51:475-483; Price R L, et al. Journal of Biomedical Materials Research Part A 2003; 67A:1284-1293; Webster T J, et al. Biomaterials 2004; 25:4731-4739; Park G E, et al. Biomaterials 2005; 26:3075-3082; Thapa A, et al. Journal of Biomedical Materials Research Part A 2003; 67A:1374-1383; Christenson E M, et al. Journal of Orthopaedic Research 2007; 25:11-22.) These properties make nanomaterials ideally suited to enhance the biocompatibility and cell/tissue interaction with extracellular matrix-derived scaffolds.

The surface energy increase caused by the addition of nanoparticles is measured as compared to an otherwise identical biocomposite having micron-sized structures. Also, this surface energy increase is evidenced by increased protein adsorption as compared to an otherwise identical biocomposite having micron-sized structures. The identical biocomposite having micron-sized structures has the same matrix and chemical identity of the particles crosslinked to the matrix, but instead of nano-sized particles, rods, fibers, or wires, the composite has micron-sized particles, rods, fibers, or wires. The micron-sized material has a diameter or all dimensions of at least 1000 nm. The protein adsorption can be measured by hematoxylin and eosin (H&E) stain of the composite followed by histology reading to quantify the amount of proteins adsorbed to the composition.

The first nanomaterials are capable of promoting tissue ingrowth and can be selected from a variety of nanomaterials that are nontoxic and biocompatible such as gold, silver, silicon carbide, degradable polymers (polylactic acid/polyglycolic acid, polycaprolactone), carbon nanotubes, silicon, silica and combinations thereof.

The first nanomaterial can be a gold nanoparticle, a gold nanorod, a gold nanofiber, a silver nanoparticle, a silver nanorod, a silver nanofiber, a platinum nanoparticle, a platinum nanorod, a platinum nanofiber, a titania nanoparticle, a titania nanorod, a titania nanofiber (rutile structure, Ti₂O₃, BaTiO₃, and the like), a silicon nanoparticle, a silicon nanorod, a silicon nanofiber, a silica nanoparticle, a silica nanorod, a silica nanofiber, an alumina nanoparticle, an alumina nanorod, an alumina nanofiber, a BaTiO₃ nanoparticle, a BaTiO₃ nanorod, a BaTiO₃ nanofiber, a polycaprolactone nanofiber, a polyglycolic acid nanofiber, a polylactic acid nanofiber, a polylacticglycolic acid nanofiber, a polydoxanone nanofiber, a trimethylene carbonate nanofiber, or a combination thereof.

Various preferred nanomaterials are a gold nanoparticle, a gold nanorod, a gold nanofiber, a silver nanoparticle, a silver nanorod, a silver nanofiber, or a combination thereof. The nanomaterials can have a mean diameter from about 5 nm to about 500 nm; from about 15 nm to about 300 nm; from about 15 nm to about 250 nm; from about 20 nm to about 150 nm; or from about 80 nm to about 120 nm.

Further, the nanorods, nanowires, or nanofibers can have a mean length of from about 100 nm to about 20 μm; from about 500 nm to about 20 μm; from about 1 μm to about 10 μm; or about 10 μm.

The second nanomaterial can be an amorphous calcium phosphate, a hydroxyapatite, a bioactive glass, a zirconia, a zirconium (IV) oxide, a calcium oxide, an aluminum oxide, a zinc oxide or a combination thereof.

The second nanomaterial comprises an amorphous calcium phosphate, a hydroxyapatite, or a combination thereof. Preferably, the second nanomaterial comprises hydroxyapatite.

The second nanomaterial can have a mean diameter from about 5 nm to about 500 nm. Preferably, the mean diameter is from about 20 nm to about 200 nm; from about 50 nm to about 150 nm; or from about 50 nm to about 130 nm.

Further, the particle sizes for the nanoparticles can be polydisperse or monodisperse. When gold nanoparticles are used, the nanoparticles are monodisperse. Such a diameter for the nanoparticles provides a specific surface area of from about 8.6×10⁴ cm²/g to about 3.5×10⁵ cm²/g; from about 1×10⁵ cm²/g to about 2×10⁵ cm²/g or about 1.5×10⁵ cm²/g. These specific surface areas are for one nanoparticle, thus, the combined specific surface are of several nanoparticles in the nanocomposite would be the specific surface area of one nanoparticle multiplied by the density of the nanoparticles in the nanocomposite.

In the functionalizing step, the selected nanomaterials obtained commercially or synthesized according to various procedures in the field can be exposed to a plasma environment with selected plasma chemistry in order to introduce new functionalities which will enhance the bonding between the nanomaterials and tissue. Generally, the precursor selected for plasma polymerization is a molecule that has one or more of the desired functional groups and one or more carbon-carbon double bonds. For example, if the desired surface functional group is an amine, the precursor would contain an amine and a carbon-carbon double bond. Examples of amines that can be used in plasma polymerization are allylamine, poly(allylamine), diaminocyclohexane, 1,3-diaminopropane, heptylamine, ethylenediamine, butylamine, propargylamine, propylamine, and the like. Amines that can be used in plasma polymerization are poly(allylamine), diaminocyclohexane, 1,3-diaminopropane, heptylamine, ethylenediamine, butylamine, propargylamine, propylamine, and the like.

When the desired surface functional group is a carboxylic acid, the precursor would contain a carboxylic acid group and a carbon-carbon double bond. Examples of compounds used are acrylic acid, methacrylic acid, propanoic acid, and the like. When the desired surface functional group is a hydroxyl group, the precursor would contain a hydroxyl group and a carbon-carbon double bond. Examples are allyl alcohol, hydroxyethyl methacrylate, hydroxymethyl acrylate, hydroxybutyl methacrylate, and the like.

The functional groups, such as —NHx (x=1 or 2), —OH, —COOH, can be selected to act as anchoring points for crosslinking the decellularized tissue substrate via covalent bond formation. A variety of plasma chemistry may be employed to introduce the functional groups. For example, allylamine may be used to deposit —NH, and —NH₂ containing plasma coatings on the nanomaterial surfaces. Allyl alcohol, hydroxyethyl methacrylate (HEMA), acrylic acid, methacrylic acid, hydroxymethyl acrylate, hydroxybutyl methacrylate, or a combination thereof may be utilized as the monomers to deposit plasma coatings and introduce —OH, —COOH functional groups on nanomaterial surfaces. Additionally, organosilicons including trimethylsilane (3MS) and hexa-methyldisiloxane (HMDSO) may be used to plasma coat the nanomaterials to ensure excellent adhesion of plasma coating to nanowires. The organosilicon coating provides a layer on the nanomaterial that aids adhesion of the nanoparticle to the deposited functionalized coating. Subsequent plasma treatment using O₂ or CO₂ may be used to further increase the surface concentration of these functional groups.

Furthermore, nanomaterials may be functionalized via a chemical reaction utilizing an activating agent (e.g., an agent capable of activating a carboxylic acid); for example, dicyclohexyl carbodiimide, diisopropylcarbodiimide, or ethyl dimethylaminopropylcarbodiimide. The activating agent can be used alone or in combination with an agent that improves efficiency of the reaction by stabilizing the reaction product; one stabilization agent is NHS (N-hydroxysuccinimide). EDC (1-ethyl-3-[3-dimethylaminopropyl]carbodiimide) and NHS (N-Hydroxysuccinimide) are used as crosslinking agents wherein EDC reacts with the carboxylic acid groups found on nanomaterials such as degradable polymers and forms an O-acrylisourea derivative and NHS stabilizes this derivative and forms a succinimidyl ester bond, which allows binding to an amino group of the tissue by forming a covalent peptide bond with the nanomaterial. When EDC and NHS are used to functionalize the nanomaterials, the molar ratio of the agents range from about 1:5 EDC:NHS to about 5:1 EDC:NHS; or about 2:5 EDC:NHS. Alternatively, nanomaterials may be functionalized via aminolysis by ethylenediamine or N-Aminoethyl-1,3-propanediamine.

For the preferred first nanomaterials of gold nanoparticles, gold nanorods, gold nanofibers, silver nanoparticles, silver nanorods, silver nanofibers, or combinations thereof, the nanomaterials can be functionalized by coordinating a ligand containing the desired functional group to the gold or silver atom. Generally, the ligand should have at least two functional groups; one of the functional groups can coordinate to the metal site and the other could be used to crosslink with the decellularized material. For example, a ligand having a thiol group and an amine group; e.g., cysteine, methionine, mercaptoalkylamines such as mercaptomethylamine, mercaptoethylamine (MEA), mercaptopropylamine, mercaptobutylamine, and the like, can be coordinated to the metal of the nanomaterial to provide a functional group for further reaction with the decellularized material. Also, a ligand having a thiol group and a carboxylic acid group; e.g., thiosalicylic acid, 2-mercaptobenzoic acid, can be coordinated to the metal of the nanomaterial to provide a functional group for further reaction with the decellularized material.

When the nanomaterial is silicon carbide, the silicon carbide nanomaterial can be treated with various reagents that have at least two functional groups; one group that can react with the surface hydroxy groups on the silicon carbide and another functional group that can crosslink to the decellularized material. For example, the silicon carbide particles can be reacted with aminoalkyl-trialkoxysilanes such as aminomethyl-trimethoxysilane, aminoethyl-trimethoxysilane, aminopropyl-trimethoxysilane, aminobutyl-trimethoxysilane, aminomethyl-triethoxysilane, aminoethyl-triethoxysilane, aminopropyl-triethoxysilane, aminobutyl-triethoxysilane, aminomethyl-tripropoxysilane, aminoethyl-tripropoxysilane, aminopropyl-tripropoxysilane, aminobutyl-tripropoxysilane, aminomethyl-tributoxysilane, aminoethyl-tributoxysilane, aminopropyl-tributoxysilane, aminobutyl-tributoxysilane, or a combination thereof to provide amine groups on the surface of the silicon carbide nanomaterial.

The functionalization of the gold nanoparticles produces nanoparticles that have from about 1×10⁻¹⁰ mol/cm² to about 1×10⁻⁹ mol/cm²; from about 2×10⁻¹⁰ mol/cm² to about 1×10⁻⁹ mol/cm² or from about 5×10⁻¹⁰ mol/cm² to about 1×10⁻⁹ mol/cm² functional groups per gold nanoparticle.

Nanomaterials that have reactive surface groups like the hydroxyl groups of the hydroxyapatite nanomaterials can be unfunctionalized upon crosslinking with the decellularized tissue substrate.

Optionally, in addition to the endogenous proteins, growth factors, and peptides that enhance cell adhesion, cell growth, and cell infiltration into the implant material, the functionalization step may include a substep to increase tissue integration, whereas the nanomaterials may be treated with exogenous cell adhesion proteins and/or peptides. The addition of these active group will promote better cellular adhesion, vascularization, and improve overall biocompatibility. The ECM proteins are important in cell adhesion. Cell adhesion to ECM proteins is mediated by integrins. Integrins bind to specific amino acid sequences on ECM proteins such as RGD (arginine, glycine, aspartic acid) motifs. Therefore there has been research conducted on the control of the orientation and conformation of cell adhesion proteins onto materials so that RGD motifs are accessible to integrins. For example, fibronectin and fibronectin-III have been adsorbed onto synthetic surfaces. The results showed that presence of fibronectin-III displayed more cell-binding domains than the fibronectin-free surface. Thus, it is possible to manipulate and specifically orient the cell binding proteins so that increased tissue integration is possible. Another in vivo study by Williams et al. (S. K. Williams, et al. Covalent modification of porous implants using extracellular matrix proteins to accelerate neovascularization. J Biomed Mater Res. 78A: 59-65, 2006) analyzed collagen type IV, fibronectin, and laminin type I's ability to promote peri-implant angiogenesis and neovascularization. Laminin stimulated extensive peri-implant angiogenesis and neovascularization into the porous ePTFE substrate material.

Additionally, vascular endothelial growth factor (VEGF) is a chemical signal secreted by cells to stimulate neovascularization. VEGF stimulates the proliferation of endothelial cells. TGF-B1 (transforming growth factor) is another chemical signal that stimulates the differentiation of myofibroblasts. Both types of growth factors have been incorporated into tissue engineered scaffolds and can be incorporated into the nanocomposites described herein to stimulate and accelerate reconstitution of native tissue.

Additional amines on the functionalized nanomaterials can be used as sites for attaching cell adhesion peptides, growth factors, glycosaminoglycans, or anti-inflammatory medications to further improve the biocompatibility of the scaffold.

### Crosslinking Nanomaterial to Autograft or Decellularized Tissue Substrate

Crosslinking of the nanomaterial to the autograft or decellularized tissue substrate is joining the two components by a covalent bond. Crosslinking reagents are molecules that contain two or more reactive ends capable of chemically attaching to specific functional groups on proteins or other molecules (e.g., decellularized tissue substrate). These functional groups can be amines, carboxyls, or sulfhydryls on the decellularized tissue substrate. To react with amines in the tissue, the crosslinking agent is selected from N-hydroxysuccinimide ester (NHS ester), N-gamma-maleimidobutyryloxy succinimde (GMBS), imidoester (e.g., dimethyl adipimidate, dimethyl pimelimidate, dimethylsuberimidate, dimethyl 3,3″-dithiobispropionimidate.2 HCl (DTBP)), pentafluorophenol ester (PFP ester), hydroxymethyl phosphine. A carboxyl group on the tissue can react with an amine on the nanoparticle directly by activation with carbodiimide. Various carbodiimides can be used including 1-ethyl-3-(3-dimethylaminopropyl)carbodiimide, dicyclohexyl carbodiimide, diisopropylcarbodiimide, and the like. A sulfhydryl group on the tissue can react with a malemide (e.g., N-e-Maleimidocaproic acid (EMCA)), haloacetyl (e.g., SBAP (NHS ester/bromoacetyl), SIA (NHS ester/iodoacetyl), SIAB (NHS ester/iodoacetyl), Sulfo-SIAB (sulfo-NHS ester/iodoacetyl), pyridyldisulfide (1,4-di(3′-(2′-pyridyldithio)-propionamido)butane (DPDPB), sulfosuccinimidy 6-(3′-[2-pyridyldithio]-propionamido)hexanoate (Sulfo-LC-SPDP), N-[4-(p-azidosalicylamido)butyl]-3′-(2′-pyridyldithio)propionamide (APDP)), or vinyl sulfone.

To enhance the crosslinking between the selected nanomaterials and autograft substrate or decellularized tissue substrate, the functionalized nanomaterials with surface functional groups capable of bonding with tissue are preferred over the “naked” nanomaterials. Though a variety of functional groups may be selected, various functional groups that are capable of forming covalent peptide bonding with tissue, such as —NH, —NH₂, —COOH, or a combination thereof, are employed.

In the crosslinking step, depending on the surface functional groups introduced, the functionalized nanomaterials are incubated (or mixed) with the autograft or decellularized tissue substrates in a crosslinking solution via a crosslinking procedure available or known to the researchers in the field. The crosslinking agent can be N-gamma-maleimidobutyryloxy succinimde (GMBS), N-e-Maleimidocaproic acid (EMCA), and Dimethyl 3,3′-dithiobispropionimidate.2 HCl (DTBP). For example, the crosslinking solution may contain acetone, 1×PBS (phosphate buffered saline), EDC (1-ethyl-3-[3-dimethylaminopropyl]carbodiimide) and NHS (N-Hydroxysuccinimide). For the crosslinking reaction, a tissue:solution volume ratio of from about 1:100 to about 20:100; from about 5:100 to about 15:100; from about 7:100 to about 10:100; or an 8:100 ratio is maintained and for rinsing, a tissue:solution volume ratio from about 0.1:100 to about 10:100; from about 0.5:100 to about 2:100; or 1:100 ratio is maintained for all subsequent rinses.

Various concentrations of nanomaterials may be utilized to achieve optimal crosslinking The incubation generally lasts about 24 hours at room temperature on an orbital shaker table at low rpm. Following incubation, the resulting crosslinked tissues are vigorously rinsed with 1×PBS for 48 hours on an orbital shaker table with several changes of the PBS solution to remove residual crosslinkers and unbound nanomaterials. Crosslinked tissues are then stored in 1×PBS at 4° C. until subsequent testing or sterilization occurs.

The crosslinking density in the nanocomposite can generally be measured by a collagenase assay wherein an increase in release of hydroxyproline indicates degradation of collagen. It would be expected that tissues that had lower crosslinking density would have a greater rate of collagen degradation and result in more hydroxyproline being released. Further, the mechanical properties can measure the crosslinking density wherein the tensile strength would be expected to increase with increasing crosslinking density. Further, the differential scanning calorimetry measurements indicate the crosslinking density of the material because a material that has a greater crosslinking density should have a higher denaturation temperature.

### Synthesis

The invention further provides a method for fabricating the nanocomposite. The inventive method includes three major steps 1) decellularizing a piece of pre-selected biological (may also be called natural) tissue, 2) optionally, functionalizing a pre-selected nanomaterial, and 3) crosslinking the decellularized tissue substrate with the optionally functionalized nanomaterial.

When an autograft is used, the nanocomposites are prepared by 1) optionally, functionalizing a pre-selected nanomaterial, and 2) crosslinking the autograft tissue substrate with the optionally functionalized nanomaterial.

The method comprises decellularizing a selected biological tissue to produce an oblong or elongate decellularized tissue substrate with cells and cellular remnants removed but extracellular matrix components intact; functionalizing a first nanomaterial to produce a functionalized first nanomaterial with surface functional groups capable of bonding with the decellularized tissue substrate; selecting a second nanomaterial; and crosslinking the decellularized tissue substrate with the first nanomaterial and the second nanomaterial by contacting the whole length of the oblong or elongate decellularized tissue substrate with the functionalized first nanomaterial and contacting at least one longitudinal end margin of the oblong or elongate decellularized tissue substrate to the second nanomaterial to form the oblong or elongate nanocomposite.

The decellularizing step may include a substep of selecting a piece of biological tissue, which may be obtained commercially, or harvested via either allografts or xenografts. The selected natural tissue may be cut into the desired shapes and sizes and needs to be stored in a buffered solution containing protease inhibitors and bacteriostatic agents at pH about 8 and 4° C. to prevent degradation of the tissue by lysosomal enzymes released by the biological cells.

### Uses

The inventive nanocomposite may be used in a wide range of tissue engineering applications, where the nanocomposite is made into scaffolds to repair defective tissue or to deliver cells, growth factors, and other additives to a healing site. For example, the nanocomposite can be utilized as a soft tissue repair material for such applications as ligament, tendon, and cartilage repair and replacement.

Preliminary testing indicates that nanocomposite materials possess adequate mechanical properties for many soft tissue repair applications. The testing results (discussed in detail in the example section) also show that the decellularized tissue substrate crosslinked with nanomaterials provides improved biocompatibility over the naked decellularized tissue substrate. The decellularized tissue substrate crosslinked with nanomaterials when implanted also favorably affects cellular responses.

A decellularized tissue substrate of derived from a tendon can be crosslinked with a functionalized gold nanoparticle throughout the whole length of the decellularized tendon and crosslinked with a hydroxyapatite nanoparticle that has an increased mass concentration at a longitudinal end margin. This material is well suited to repair or replace a ligament or tendon in a subject.

Having described the invention in detail, it will be apparent that modifications and variations are possible without departing from the scope of the invention defined in the appended claims.

### EXAMPLES

The following non-limiting examples are provided to further illustrate the present invention.

### Example 1

### Decellularization of Natural Tissue

Allografts were purchased from a tissue bank (Musculoskeletal Transplant Foundation, Jessup Pa.). Xenografts were harvested from the collagen rich central tendon portion of porcine diaphragm and decellularized. The harvested natural tissue was placed immediately into a tissue storage/decellularization solution comprised of Tris buffer solution (pH 8.0), 5 mM ethylenediaminetetraacetic acid (EDTA), 0.4 mM phenylmethanesulfonyl fluoride (PMSF), and 0.2% (w/v) sodium azide and stored at 4° C. until ready to use.

To make 1.0 L of tissue storage/decellularization solution, 70 mg of PMSF was dissolved in 2.0 mL of anhydrous isopropanol. The dissolved PMSF was then added to a mixture of 1 package Tris buffer, 1.86 g EDTA, and 2 mg sodium azide in 1.0 L of double distilled water. The solution was mixed well using a stir bar until dissolved (approximately 1 hour).

In the decellularization step, a 250 mL flask was first charged with 100 mL of this solution and 1 mL of tributyl phosphate (1% v/v). Next, a piece of natural tissue (5 cm×5 cm) was added to the flask, and the tissue flattened at the bottom of the flask with a forceps. The flask was placed on an orbital shaker, set to 225 rpm, and agitated continuously for 24 hours at room temperature. After 24 hours, the decellularization solution was discarded and the abovementioned sequence repeated once.

In the rinsing step, the decellularized tissue was removed from the flask and the solution discarded. The flask was washed to remove residual chemicals, then to it added 100 mL of distilled water and decellularized tissue. The flask was placed on an orbital shaker, set to 225 rpm, and agitated continuously for 24 hours at room temperature. After 24 hours, the water was discarded and 100 mL of 70% (v/v) ethanol was added. Again, the flask was agitated at 225 rpm for 24 hours at room temperature. The resulting tissue was stored in 70% (v/v) ethanol at 4° C.

### Example 2

### Crosslinking of Gold Nanoparticles (AuNP) and Hydroxyapatite Nanoparticles (HaNP) to Decellularized Tissue

Gold nanoparticles (20 to 200 nm diameter) were purchased from Fitzgerald Industries International (Concord, Mass.) and Ted Pella, Inc. in the form of a gold colloid solution. In order to functionalize the nanoparticles with terminal amino groups, a solution of 10 mg/mL of 2-mercaptoethylamine (MEA) in water was prepared. To functionalize AuNP with 15 μM of MEA, 3.4 μL of a concentrated MEA solution was added to 20 mL of gold colloid solution, and mixed well.

Hydroxyapatite nanoparticles (<200 nm in diameter) were purchased from Sigma-Aldrich Company (St. Louis, Mo.) in the form of a 10 wt. % solution in water. Prior to use, the vial containing the HaNP solution was sonicated for 1 to 4 hours at a temperature of 40-55° C. Immediately prior to use, the vial was removed from the sonicator, and placed in a beaker of ice water to cool until just slightly warmer than room temperature. Cooling and vortexing was alternated to ensure that the HaNP remained in solution, non-clumping.

A crosslinking solution comprised of a 50:50 (v/v) solution of acetone and phosphate buffered saline (pH 7.5) was prepared. To make 1.0 L of crosslinking solution, 497.50 mL of acetone and 497.50 mL of 1× phosphate buffered saline (PBS) were combined. Separately, 576 mg of N-hydroxysuccinimide (NHS) were dissolved in 2.5 mL of dimethylformamide. Also separately, 383 mg of 1-ethyl-3-(3-dimethylaminopropyl)-carbodiimide (EDC) were dissolved in 2.5 mL of MES buffer (e.g., 0.1 M MES (2-(N-morpholino)ethanesulfonic acid in 0.5 M aqueous NaCl at pH 6.0). The EDC and NHS solutions were immediately mixed together, and then added to the acetone/PBS solution.

The decellularized tissue was incubated in 50 mL of the crosslinking solution for 15 minutes at room temperature, without shaking Control grafts were incubated in 10 to 50 mL of crosslinking solution overnight at room temperature without shaking This tissue was crosslinked with EDC/NHS but not conjugated to nanoparticles.

After the incubation period, the tissue was placed in a solution of 2 to 5 mL of crosslinking solution in a disposable Petri dish (100 mm×15 mm). Enough solution was present to hydrate the tissue without flowing over the top of the tissue and washing off nanomaterials.

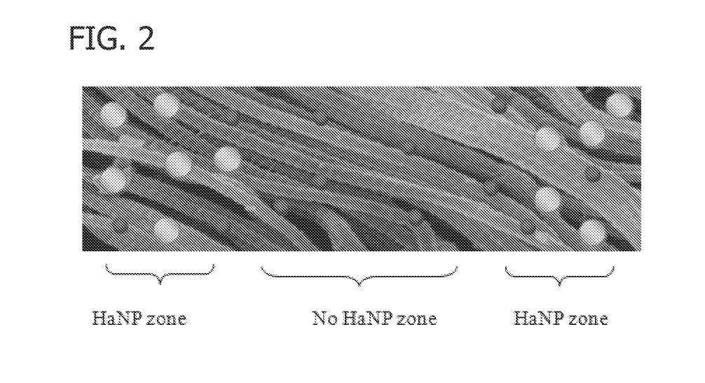

In the conjugation step, 1 to 5 mL of functionalized AuNP in MEA was pipetted on top of the tissue, followed by 1 to 5 mL of the just vortexed solution of HaNP. The AuNP solution was evenly distributed on top of the tissue. The HaNP solution was added to both ends of the tissue but not to the middle of the tissue (See FIGS. 1 and 2). The treated tissue was then incubated for 12 to 24 hours at room temperature without shaking Following incubation, the tissue was transferred to a flask and to it was added 100 mL of 1×PBS buffer. The flask was agitated for 24 hours at room temperature at 225 rpm. After 24 hours, the PBS solution was discarded and this step repeated once more using another 100 mL of 1×PBS solution. Finally, the PBS solution was discarded and the graft tissue stored in 70% (v/v) ethanol at 4° C.

### Example 3

### Crosslinking of Silver Nanoparticles and Silver Nanowires to Decellularized Tissue

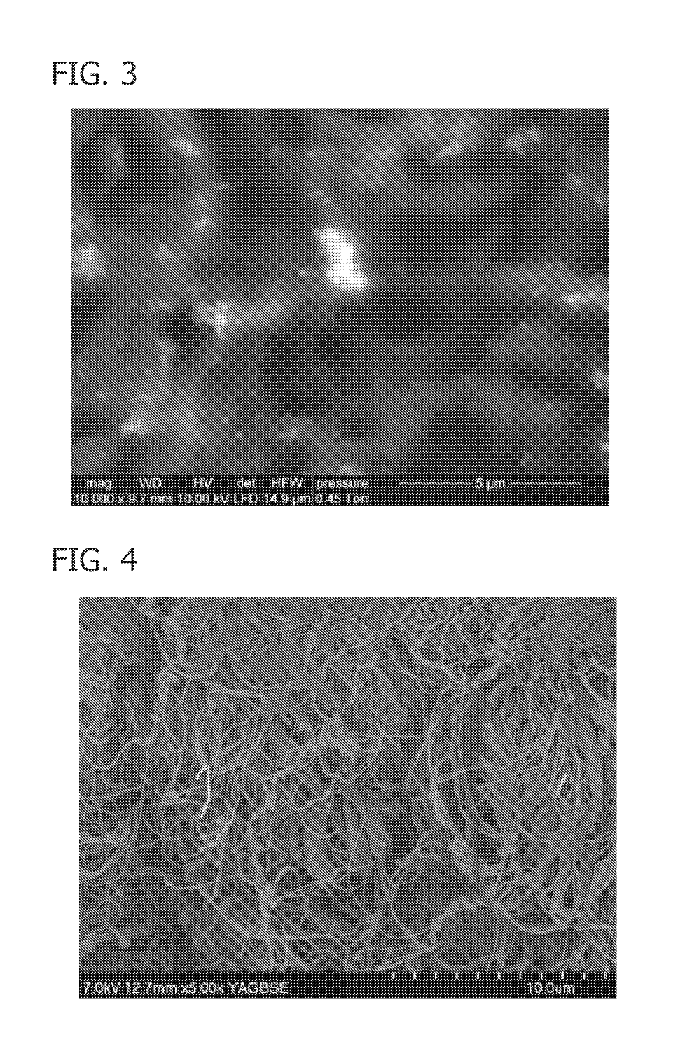

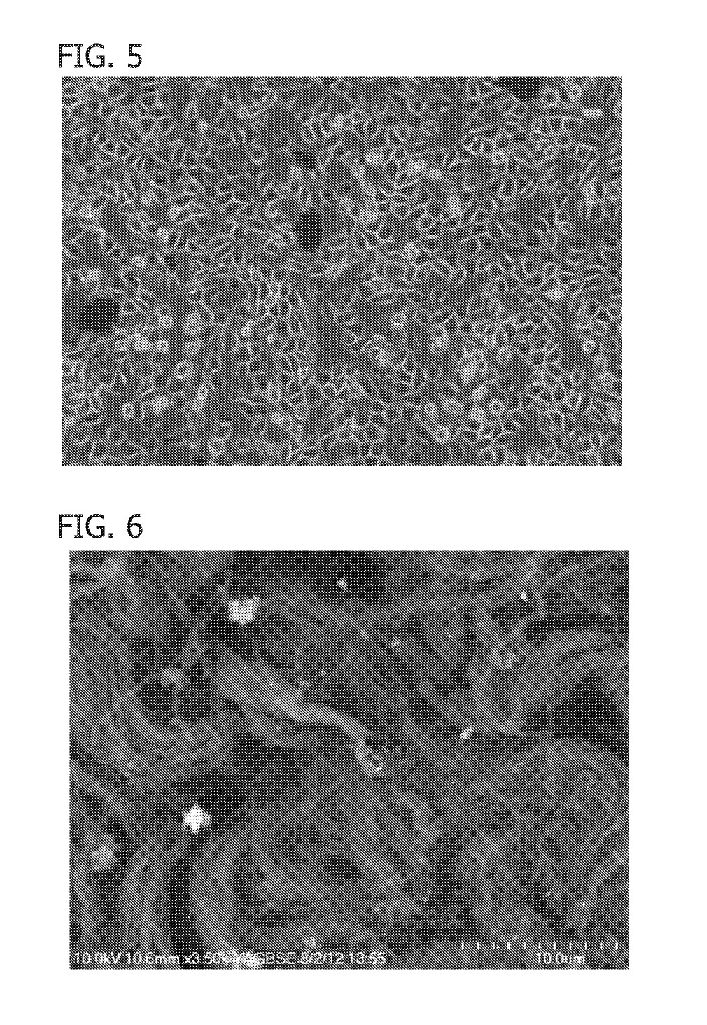

Using the procedure described in Examples 1 and 2 silver nanoparticles, silver nanowires, and calcium oxide nanoparticles were crosslinked to decellularized tissue. The SEM of the silver nanoparticles and silver nanowires crosslinked with decellularized tissue are shown in FIGS. 3 and 4, respectively. The SEMs show that the silver nanoparticles and silver nanowires conjugate well to the decellularized tissue. FIG. 5 represents a biocompatibility study for calcium oxide nanoparticles incubated with L929 fibroblast cells. After 72 hours, the cells were growing (as shown in FIG. 5) and surrounding the calcium oxide nanoparticles and indicated the calcium oxide nanoparticles were biocompatible.

### Example 4

### Preparation of Nano-Grafts

Using the protocols outlined in Examples 1 and 2, a total of 150 grafts were prepared, including 100 nano-grafts and 50 control grafts. Some of the grafts were tested for the physicochemical properties (SEM and DSC) and some for biocompatibility, both in vitro and in vivo tests. The grafts in the table below were made according to the procedure described in Examples 1 and 2.

FIG. 6 shows a representative SEM of a decellularized graft conjugated to both HaNP and AuNP.

| | | Nanoparticle |
|---|---|---|
| Tissue source | Nano-graft type (particle size) | concentration |
| Allograft | Control | 0 mL/0 mL |
| Allograft | HaNP (<200 nm) | 1.0 mL/1.5 mL |
| Allograft | AuNP (100 nm) | 1.0 mL/1.5 mL |
| Allograft | HaNP-AuNP (100 nm) | 1.0 mL/1.5 mL |
| Xenograft | Control | 0 mL/0 mL |
| Xenograft | HaNP (<200 nm) | 1.0 mL/1.5 mL |
| Xenograft | AuNP (20 nm) | 1.0 mL/1.5 mL |
| Xenograft | AuNP (100 nm) | 1.0 mL/1.5 mL |
| Xenograft | HaNP-AuNP (100 nm) | 1.0 mL/1.5 mL |

### Example 5

### Quant-iT Double Stranded DNA Quantification Assay

The Quant-iT PicoGreen® double stranded DNA (quantification assay (Life Technologies) was utilized for the cellularity studies. PicoGreen® is a cyanine dye that exhibits >1000-fold fluorescence enhancement upon binding to double stranded DNA (dsDNA).

Grafts were rinsed in sterile PBS for 24 hours and then incubated in EMEM culture medium (Eagle's Minimum Essential Medium) for 24 hours at 37° C. in a 5% CO₂ atmosphere. Grafts were then placed in 48-well cell culture plates, with one graft added per well.

A cell suspension containing approximately 3×10⁵ cells L929 Murine Fibroblast cells was removed from the culture flask and pipetted on top of each graft. The grafts were then incubated at 37° C. in a 5% CO₂ atmosphere for 30 minutes. Media was added to each well to reach a total 1 mL of solution and then the culture plate was placed in the incubator at 37° C. in a 5% CO₂ atmosphere for 3, 7, 10, or 14 days, depending on the experiment.

Grafts were moved to a new culture plate on day 2 so than any cells growing at the bottom of the first plate did not use up the media, and 1 mL of media was added to each well. Media was pipetted off every other day and replaced with 1 mL of fresh media. At the end of the incubation period, grafts were removed from the wells and rinsed with 0.5 mL of sterile PBS.

After culture, grafts were placed in individual 1.5 mL microcentrifuge tubes with a 18 gauge hole in the lid. The microcentrifuge tubes were sealed in a 50 mL centrifuge tube, and stored at −70° C. until ready to lyophilize.

Samples were lyophilized until completely dry (about 20 hours), and then the dry mass of the grafts was determined. Each graft was placed in a new sterile microcentrifuge tube using sterile forceps and to the tube was added 0.5 mL of papain digestion buffer (125 μL/mL papain in PBE buffer comprised of sterile 1×PBS, 5 mM cysteine-HCl, and 5 mM disodium EDTA at a pH of 6.15). The grafts were incubated for 24 hours at 60° C., vortexing the digests at least once during the digestion process. Samples were then centrifuged at 10,000 G for 5 minutes.

For the dsDNA assay, 75 mL of 1×TE buffer was prepared by diluting 3.75 mL of 20× TE buffer (supplied with the Quant-iT PicoGreen® ds DNA kit) in 71.25 mL of sterile, distilled, DNase-free water. PicoGreen® reagent was diluted 200-fold by placing 200 μL of reagent in 39.8 mL of 1× TE buffer.

For the assay, about 25 μL of digest solution were transferred to a 1.5 mL cuvette along with 225 μL of 1× TE buffer and 250 μL of diluted PicoGreen® reagent. The samples were incubated for 2 to 5 minutes at room temperature, away from light.

Fluorescence measurements were determined using a FluoroMax-3 Spectrofluorometer (Horiba Scientific) spectrophotometer. Samples were excited at 480 nm and fluorescence emission intensity was measured at 520 nm. Standards were prepared as follows:

| Volume | Volume (μL) | Volume (μL) | Final DNA Concentration |
|---|---|---|---|
| (μL) | of 2 μg/mL | of Diluted Quant-iT | in Quant-iT PicoGreen |
| of TE | DNA Stock | PicoGreen Reagent | Assay (ng/mL) |
| 0 | 500 | 500 | 1000 |
| 450 | 50 | 500 | 100 |
| 495 | 5 | 500 | 10 |
| 499.5 | 0.5 | 500 | 1 |
| 500 | 0 | 500 | blank |

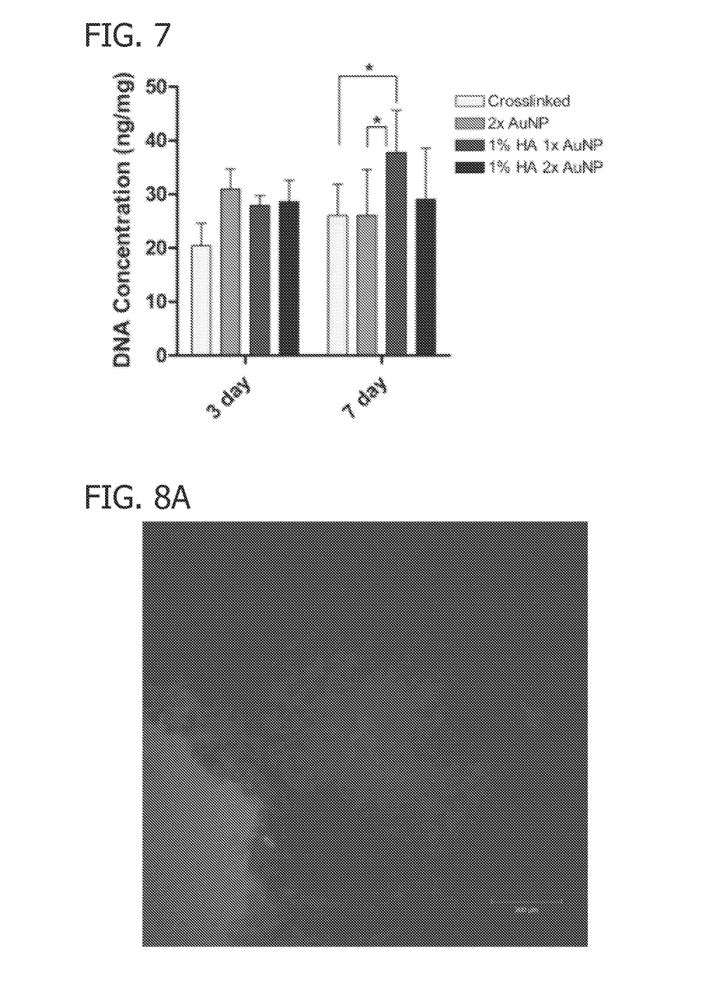

Using the Lambda DNA standard curve, the DNA concentration of the experimental samples was determined. The DNA concentration was then normalized by dividing the dry mass of the corresponding samples. Data was plotted in terms of DNA content of the nanograft (ng/mg dry weight) versus time of incubation. As shown in FIG. 7, by day 7 the 1% HaNP/1×AuNP graft had significantly more DNA per dry weight than the control crosslinked graft (p<0.05). This data provides evidence that the nanoparticles are acting as anchoring sites to increase cellular ingrowth and attachment on the graft.

### Example 6

### WST-1 Cell Assay

A second assay used for cellularity studies was the WST-1 assay (Roche Applied Science), which provides a colorimetric assay for the quantification of cell viability and proliferation. The reagent is a sterile, ready-to-use solution that contains WST-1 and an electron coupling reagent diluted in phosphate buffered saline. WST-1 is a water soluble tetrazolium salt that is reduced to a purple-colored formazan by cellular enzymes; the amount of formazan formed directly correlates to the number of metabolically active cells in the culture

Grafts were rinsed in sterile PBS for 24 hours and then placed in a BD Falcon cell culture treated 48-well plate, with one graft added per well. Grafts were incubated in 0.5 mL of EMEM culture medium (Eagle's Minimum Essential Medium) supplemented with 10% (v/v) horse serum and PennStrep (200 U/mL) for 24 hours at 37° C. in a 5% CO₂ atmosphere.

The culture medium was then removed and the grafts were treated with 1 mL of L929 murine fibroblast cell suspension containing 3×10⁴ cells/mL, then incubated for 2 days at 37° C. in a 5% CO₂ atmosphere. After 2 days, 0.5 mL of culture medium was removed and replaced with 0.5 mL of fresh culture medium. Incubation was continued for 1 day at 37° C. in a 5% CO₂ atmosphere.

To perform the assay, 0.5 mL of cell media were withdrawn from the cells and 50 μL of WST-1 reagent was added to each well, followed by incubation for 4 hours. Next, 100 μL of media from each well was placed in separate wells of a 96-well plate.

Absorbance measurements were taken using a BioRad 680 Microplate Reader. Absorbance was measured at 450 nm, with a reference filter at 655 nm. The absorbance of a control well containing culture medium was determined in order to subtract background absorbance from the experimental samples. The absorbance values were then analyzed to determine cell proliferation compared to the control well.

### Example 7

### In Vivo Implant Study in Swine

An in vivo study was conducted using transgenic swine expressing green fluorescence protein (GFP). The GFP host cells, as they populate and replace the grafts, can be tracked using fluorescence microscopy. This provides qualitative and quantitative measures of the remodeling pathways. Twelve GFP expressing swine (10 female and 2 male purchased from Dr. Randall Prather at the University of Missouri) were utilized with 6 implants each for a 1, 3, and 6 month studies (4 pigs/time point).

In this study, each pig received one of the following: crosslinked allograft (human anterior tibialis tendon), crosslinked xenograft (pig diaphragm), AuNP-allograft, AuNP-xenograft, HaNP-AuNP allograft, and HaNP-AuNP xenograft. Animal Care and Use Committee (ACUC) protocol for the treatment of pigs was followed before, during, and after surgery. The grafts were harvested at T=1 month to perform histological analysis. At the time of sacrifice, full-thickness sections of the abdominal wall, including all four repair sites and 1 cm of surrounding tissue, were harvested.

Histological analysis was done using hematoxylin and eosin (H&E) stain. Hematoxylin is a dark purplish dye that stains the chromatin within the nucleus a deep purplish-color. Eosin is an orangish-pink to red dye that stains the cytoplasmic material including connective tissue and collagen, and leaves an orange-pink counterstain. The H&E specimens were assessed microscopically for the following: cellular infiltration, inflammation/foreign body reaction, extracellular matrix (ECM) deposition, scaffold degeneration, fibrous encapsulation, and neovascularization. A modified four-point grading scale derived from Valentin et al. (Valentin, J. E., Badylak, J. S., McCabe, G. P., Badylak, S. F., J Bone Joint Surg Am, 2006, 88: 2673-2688) was utilized to quantify the results. Table 1 displays the four-point grading scale.

TABLE 1
Four point grading scale for histological analysis

| Score | 0 | 1 | 2 | 3 |
|---|---|---|---|---|
| Cellular infiltration | Zero cells in contact | Cells contact periphery, | Cells infiltrate | Cells penetrate into |
| | with scaffold | no penetration into | scaffold, but none | center of scaffold |
| | | scaffold | reach center | |
| Cell types (inflamatory | Inflammatory cells | Primarily inflammatory | Primarily fibroblasts, | Fibroblasts only, no |
| cells-neutrophils, | present, no fibroblasts | cells, few fibroblasts | few inflammatory cells | inflammatory cells |
| macrophages, foreign | | | | |
| body giant cells) | | | | |
| Host extracellular | No host ECM | Host ECM deposited | Host ECM deposited | Host ECM deposited |
| matrix deposition | deposition | at periphery of | inside scaffold, but | inside scaffold, |
| | | scaffold | not at the center | plus the center |
| Scaffold degradation | Original scaffold | Scaffold partially | Scaffold degraded, | Scaffold completely |
| | intact, borders | degraded, layers | hard to distinguish | degraded, no evidence |
| | clearly demarcated | separated by cells, | scaffold from | of original scaffold |
| | | BV, host tissue, etc. | host tissue | |
| Fibrous encapsulation | Extensive encapsulation | Moderate encapsulation | Mild encapsulation | No fibrous |
| | (50-100% of periphery) | (25-50% of periphery) | (<25% of periphery) | encapsulation |
| Neovascularization | Zero blood vessels | Vessels present at | Vessels infiltrate | Vessels penetrate into |
| | present | scaffold periphery, | scaffold but none | center of scaffold |
| | | no penetration | reach center | |

Tissue specimens were also analyzed by confocal fluorescence microscopy (CFM). In this procedure, the samples were illuminated by a tightly focused laser beam that resulted in excitation of fluorophores within the sample. The host, i.e., the GFP pig tissue fluoresces green and the specimen appears black unless there are migrating host cells into the grafts. The excited molecules fluoresce light that is collected by the microscopic objective and imaged onto the detector though filters to provide 3D optical resolution.

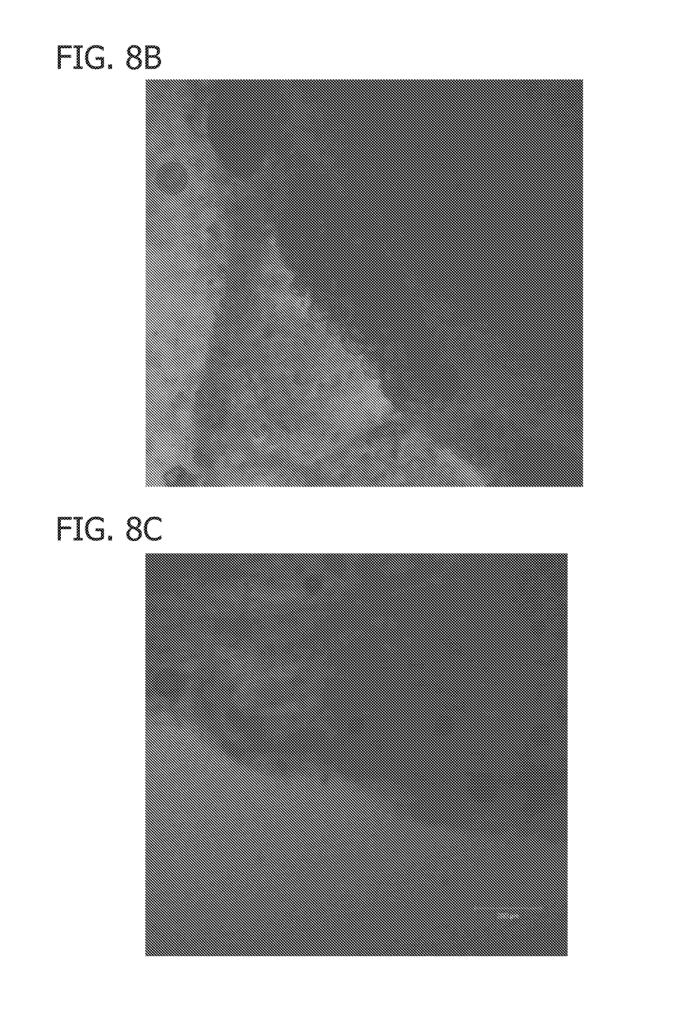

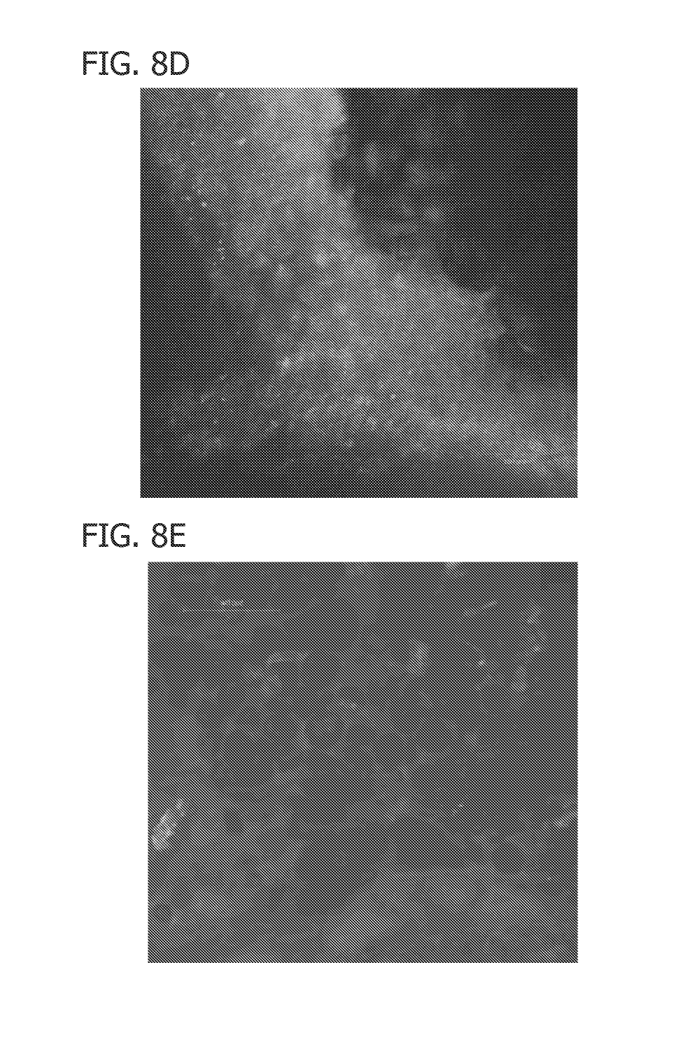

CFM images of grafts implanted for 1 month in GFP expressing swine are shown in FIGS. 8A-8E. These figures show cross-sectional slices of a cross-linked allograft (FIG. 8A), a crosslinked xenograft (FIG. 8B), a hydroxyapatite nanoparticle and gold nanoparticle (HaNP/AuNP)-allograft (FIG. 8C), a second view of a HaNP/AuNP-allograft (FIG. 8D), and an AuNP-allograft (FIG. 8E). Graft materials appear black in the images, while the host tissue appears fluorescent green.

As shown in FIG. 8A, the graft without the nanoparticles appears to have little fluorescence. In this particular view, the graft was interfaced to the subcutaneous tissue which was removed during harvesting, so that the fluorescence shown in FIG. 8A is actually free GFP that was released from the cells. This image is a longitudinal view of the graft slice, imaged at a depth of ˜50 μm to avoid the free GFP. FIG. 8B is a crosslinked graft interfaced to the muscle/fascia side (cross-sectional slice). Again, there appears to be very little integration of the host tissues into the graft. Two different HaNP/AuNP-allografts (from two different pigs) are shown in FIGS. 8C and 8D. FIG. 8C is the graft interfaced on the subcutaneous side (which is removed) and FIG. 8D is the graft interfaced on the muscle/fascia side. In both images, there appears to be in-growth of the GFP cells. FIG. 8E is a cross-sectional slice of a AuNP-allograft. GFP cells have infiltrated into the graft material. The nanomaterials appear to be enticing more in-growth as shown by the increase in fluorescence of the nanomaterial-grafts. When the GFP host material in-grows into the non-GFP grafts, fluorescent “cellular highways” are noted. This in vivo study thus demonstrates the ability of the nanomaterials to attract cells.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawing shall be interpreted as illustrative and not in a limiting sense.

1. An oblong or elongate nanocomposite comprising:
an oblong or elongate decellularized tissue substrate having a length, first and second longitudinal end margins, and a central longitudinal portion intermediate the first and second longitudinal end margins, the decellularized tissue substrate including extracellular matrix components and being substantially free from cells and cellular remnants;
a first nanomaterial crosslinked with the decellularized tissue substrate and adapted to promote tissue ingrowth, the first nanomaterial having a substantially uniform distribution of mass concentration along the length of the decellularized tissue substrate; and
a second nanomaterial crosslinked with the decellularized tissue substrate and adapted to promote osseointegration, the second nanomaterial having a non-uniform distribution of mass concentration along the length of the decellularized tissue substrate that is non-uniform, wherein the mass concentration of the second nanomaterial is greater adjacent at least one of the first and second longitudinal end margins of the decellularized tissue substrate than at the central longitudinal portion of the decellularized tissue substrate.

2. The oblong or elongate nanocomposite of claim 1 wherein the oblong or elongate decellularized tissue substrate is derived from a tendon or ligament.

3. The oblong or elongate nanocomposite of claim 1 wherein the nanocomposite is an allograft, an autograft, a xenograft, or a combination thereof.

4. The oblong or elongate nanocomposite of claim 3 wherein the nanocomposite is an allograft.

5. The oblong or elongate nanocomposite of claim 1 wherein the concentration of the second nanomaterial is higher toward the first and second longitudinal end margins of the decellularized tissue substrate.

6. The oblong or elongate nanocomposite of claim 5 wherein the second nanomaterial is substantially absent from the central longitudinal portion of the decellularized tissue substrate.

7. The oblong or elongate nanocomposite of claim 6 wherein the first longitudinal end margin is at least 30% of the total length of the decellularized tissue substrate measured from the first longitudinal end of the decellularized tissue substrate.

8. (canceled)

9. The oblong or elongate nanocomposite of claim 6 wherein the central longitudinal portion comprises at least 40% of the total length of the decellularized tissue substrate and is symmetrical about a longitudinal center of the decellularized tissue substrate.

10.-11. (canceled)

12. The oblong or elongate nanocomposite of claim 9 wherein the decellularized tissue substrate retains 1 to 10 cell nuclei or cell remnants in a 1 cm² sample.

13.-16. (canceled)

17. The oblong or elongate nanocomposite of claim 9 wherein the decellularized tissue substrate is human, porcine, bovine, or equine.

18. The oblong or elongate nanocomposite of claim 9 wherein decellularized tissue substrate comprises decellularized porcine diaphragm tendon tissue.

19. The oblong or elongate nanocomposite of claim 18 wherein the decellularized tissue substrate has a pore size from about 1 μm to about 180 μm and a porosity from about 35% to about 90%.

20.-21. (canceled)

22. The oblong or elongate nanocomposite of claim 9 wherein the first nanomaterial comprises gold, silver, silicon carbide, polylactic acid/polyglycolic acid, polycaprolactone, carbon nanotubes, silicon, silica, or combinations thereof.

23.-32. (canceled)

33. The oblong or elongate nanocomposite of claim 22 wherein the second nanomaterial comprises an amorphous calcium phosphate, a hydroxyapatite, a bioactive glass, a zirconia, a zirconium (IV) oxide, a calcium oxide, an aluminum oxide, a zinc oxide, or a combination thereof.

34. (canceled)

35. The oblong or elongate nanocomposite of claim 33 wherein the second nanomaterial comprises hydroxyapatite.

36.-43. (canceled)

44. The oblong or elongate nanocomposite of claim 33 wherein the first nanomaterial or the second nanomaterial is crosslinked with the decellularized tissue substrate using 1-ethyl-3-[3-dimethylaminopropyl]carbodiimide) and N-hydroxysuccinimide.

45.-46. (canceled)

47. A method for treating a soft tissue injury comprising implanting an oblong or elongate nanocomposite of claim 1 at the site of the injury in a subject.

48.-53. (canceled)

54. The method of claim 47 wherein said oblong or elongate nanocomposite is placed surgically.

55. The method of claim 54 wherein the oblong or elongate nanocomposite is used for ligament repair, ligament replacement, tendon repair, tendon replacement, cartilage repair, or cartilage replacement.

56.-57. (canceled)

58. A method for producing an oblong or elongate nanocomposite, comprising
decellularizing a selected biological tissue to produce an oblong or elongate decellularized tissue substrate with cells and cellular remnants removed but extracellular matrix components intact;
functionalizing a selected first nanomaterial adapted to promote tissue ingrowth to produce a functionalized first nanomaterial with surface functional groups capable of bonding with the decellularized tissue substrate;
selecting a second nanomaterial adapted to promote osseointegration; and crosslinking the decellularized tissue substrate with the functionalized first nanomaterial and the second nanomaterial by contacting the complete oblong or elongate decellularized tissue substrate with the functionalized first nanomaterial and contacting at least one longitudinal end margin of the oblong or elongate decellularized tissue substrate with the second nanomaterial to form the oblong or elongate nanocomposite.
